(19) United States
(12) Patent Application Publication
(10) Pub. No.: US 2006/0040991 A1
(43) Pub. Date: Feb. 23, 2006

(54) Pharmaceutical presentation form for oral administration of a poorly soluble active compound, process for its preparation and kit

(75) Inventor: Berthold Roessler, Halle (DE)

Correspondence Address: SENNIGER POWERS, ONE METROPOLITAN SQUARE, 16TH FLOOR, ST LOUIS, MO, 63102, US

(73) Assignees: Baxter International Inc., Deerfield, IL (US); Baxter Healthcare S.A., Wallisellen (CH)

(21) Appl. No.: 11/169,407

(22) Filed: Jun. 29, 2005

Related U.S. Application Data
(60) Provisional application No. 60/583,815, filed on Jun. 29, 2004.

(30) Foreign Application Priority Data
Jun. 29, 2004 (DE) DE 10 2004 031 5

(51) Int. Cl. A61K 31/4439

(52) U.S. Cl. 514/339

(57) ABSTRACT

The invention relates to a pharmaceutical presentation form for the oral administration of indibulin in the form of an aqueous drink solution, and a method for its preparation.

### FIELD OF THE INVENTION

The invention relates to a pharmaceutical presentation form for oral administration of a poorly soluble active compound (D-24 851; INN: indibulin) in the form of an aqueous drink solution, a method for its preparation, and a kit for the simplified handling of the preparation.

Under the name indibulin (D-24 851) [see German patent application No. 196 36 150.8, the contents of which are incorporated by reference herein] is disclosed a novel, synthetic antitumor active compound having the chemical name N-(pyridin-4-yl)-[1-(4-chlorobenzyl)indol-3-yl]-glyoxylamide, of the empirical formula C₂₂H₁₆ClN₃O₂ and of the following structural formula:

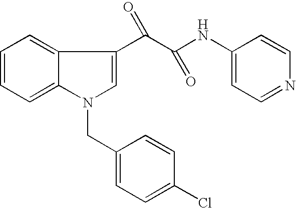

This compound is distinguished by a significant in vitro and in vivo activity as a tubulin inhibitor [see German patent applications 198 14 838.0 and 199 46 301.8, the contents of both of which are incorporated by reference herein]. Indibulin is a tubulin inhibitor which intervenes in the cell division process, stops this and thereby, in particular, effectively suppresses the growth of tumors.

Indibulin is obtained as a white crystalline powder and is virtually insoluble in hydrophilic solvents such as, for example, water, methanol, ethanol and isopropanol. Corresponding to these properties, the bioavailability of indibulin is low if it is administered perorally in customary presentation forms, e.g. as a powder, granule, tablet or capsule.

Adequate solubility is seen in various organic solvents, such as, for example, dimethylformamide, dimethyl sulfoxide and N-methylpyrrolidone. These organic solvents, however, are unsuitable for oral administration to the patient on account of their toxicity or other intolerable properties.

Highly concentrated (i.e. greater than 50%, percent by weight) organic acids, e.g. acetic acid or lactic acid, are relatively good solvents for indibulin. Highly concentrated organic acids, however, are toxic to the body and have a destructive effect on mucous membrane cells, such that oral administration is excluded.

To increase the bioavailability of poorly soluble active compounds, various formulation methods are known and have been used which correspond to the prior art.

These are:
1. Micronization of the active compound and processing to give oral presentation forms, for example, suspensions, capsules or tablets [R. Voigt, Lehrbuch der Pharm. Tech; Hagers Handbuch (Textbook of Pharm. Tech; Hager's Handbook) Volume 2, Chap. 12.2; Bauer, Frömming, Führer, Pharmazeutische Technologie (Pharmaceutical Technology)]. Formulations of this type, however, led in the case of indibulin to a comparatively low bioavailability, combined with low plasma levels and inadequate activity (see Example 2).
2. Dissolving or suspending the active compound in organic solvents and solubilizers (surfactants) [R. Voigt, Lehrbuch der Pharm. Tech; Hagers Handbuch (Textbook of Pharm. Tech; Hager's Handbook) Volume 2, Chap. 12.2; Bauer, Frömming, Führer, Pharmazeutische Technologie (Pharmaceutical Technology)]. By the use of solubilizers (see example 3), it was possible to increase the bioavailability of indibulin. However, the formulations cannot be administered to humans because of the high proportion of excipient.
3. Preparation of colloidal suspensions, nano- or microparticle suspensions. Here, using high shear forces, the active compound is finely comminuted or precipitated from a solvent mixture as fine particles. For stabilization of the suspensions, surfactants, salts and viscosity-influencing excipients are used. The preparation of this type of pharmaceutical preparations is associated with high technical expenditure in comparison to the inventive preparation.

It has now surprisingly been found that using an aqueous solution of a physiologically tolerable, organic acid, e.g. acetic acid, glycolic acid, in particular lactic acid, it is possible to prepare a supersaturated aqueous solution of the active compound which is adequately stable for administration. Surprisingly, the oral presentation form according to the invention leads, on oral administration, to an increased bioavailability of the active compound. An administrable, physiologically tolerable acid concentration of less than approximately 20% (percent by weight), in particular from approximately 5 to approximately 15% (percent by weight) can be obtained.

### SUMMARY OF THE INVENTION

According to one aspect of the invention, a pharmaceutical presentation form (or “pharmaceutical composition”, or “pharmaceutical dosage form”, or “pharmaceutical form of administration”) comprising the poorly soluble active compound indibulin for oral administration, obtainable by dissolving indibulin in or with a highly concentrated organic acid which is physiologically tolerable per se in diluted form, and subsequent dilution by addition of water to a physiologically tolerable concentration of the organic acid, is made available, indibulin being present in the form of a supersaturated solution.

Suitable embodiments of the invention are in each case described in the dependent claims.

According to a further aspect of the invention, a method for the preparation of a pharmaceutical presentation form comprising the poorly soluble active compound indibulin for oral administration is made available, wherein the active compound indibulin is optionally dissolved with shaking in a highly concentrated organic acid which is physiologically tolerable in diluted form, and subsequently the solution thus obtained is diluted to a physiologically tolerable concentration of the organic acid by addition of water, indibulin being present in the form of a supersaturated solution.

Suitable embodiments of the invention are in each case described in the dependent claims.

According to a further aspect of the invention, a kit for the simplified handling of the preparation of a pharmaceutical presentation form comprising the poorly soluble active compound indibulin for oral administration as outlined above is made available, comprising 1.) a specified amount of the active compound indibulin, 2.) a specified amount of a highly concentrated organic acid and 3.) a specified amount of water, which optionally contains a specified amount of one or more taste corrigents.

Suitable embodiments of the invention are in each case described in the dependent claims.

### BRIEF DESCRIPTION OF THE DRAWINGS

FIG. 1 is a graph of plasma level curves over 96 hours after single administration of 20 mg, 40 mg or 80 mg of indibulin in the formulation according to Example 1.

### DETAILED DESCRIPTION OF THE INVENTION

The present invention makes it possible, in an inventive manner, to prepare a supersaturated aqueous solution of the poorly water-soluble active compound indibulin, which surprisingly leads to an improved and adequate bioavailability on oral administration. This is achieved by dissolving the active compound in a highly concentrated organic acid which is physiologically acceptable per se in diluted form, preferably lactic acid (for example approximately 50% to approximately 90%, percent by weight), and subsequent dilution of the solution with water or aqueous excipient solutions, e.g. flavored glucose solution, to give a physiologically tolerable acid concentration of less than 20% by weight, preferably approximately 5% to approximately 15% by weight. A supersaturated aqueous solution having a concentration of approximately 0.2 mg/ml to approximately 2 mg/ml results here, preferably approximately 0.5 mg/ml to approximately 1.5 mg/ml, in particular approximately 1 mg/ml, of the poorly soluble active compound having a physical stability adequate for use as a drink solution of approximately 2 hours. The preparation of the drink solution is carried out with the aid of a preparation set with simple handling immediately before administration.

The preparation set (kit) consists of:
1. the poorly soluble active compound (e.g., indibulin)
2. the highly concentrated organic acid (e.g., lactic acid 90%)
3. an aqueous solution of taste ingredients (e.g., glucose and flavoring).

These three components can be present in separate containers or in a technical device for mixing the components consisting of three chambers (Example 6).

The invention is illustrated in more detail in Examples 1 to 7, without being restricted thereto.

### EXAMPLE 1

### Lactic Acid Formulation Having a Concentration of 1 mg/ml

For the preparation of the drink solution, the active compound indibulin is dissolved in lactic acid 90% European Pharmacopoeia (“Ph. Eur.”) and subsequently diluted with a solution of glucose and maracuja flavoring in water.

60 ml of an aqueous drink solution of indibulin having a concentration of 1 mg/ml are obtained. The total volume of the solution after preparation can, however, be varied at will and can be, for example, also 100 ml or 200 ml. The excipients glucose and maracuja flavoring are the taste enhancers which facilitate oral administration.

Composition of the Solution:

| | | |
|---|---|---|
| Indibulin | 60.0 | mg |
| Lactic acid 90% | 7269.2 | mg |
| Glucose Ph. Eur. | 5532.5 | mg |
| Maracuja flavoring | 96.9 | mg |
| Water (Wfi) | 50503.7 | mg |

### EXAMPLE 2

### Lactic Acid Formulation Having a Concentration of 2 mg/ml

For the preparation of the drink solution, the active compound indibulin is dissolved in lactic acid 90% Ph. Eur. and subsequently diluted with a solution of glucose and maracuja flavoring in water.

60 ml of an aqueous drink solution of indibulin having a concentration of 2 mg/ml are obtained. The exipients glucose and maracuja flavoring are the taste enhancers which facilitate oral administration.

Composition of the Solution:

| | | |
|---|---|---|
| Indibulin | 120.0 | mg |
| Lactic acid 90% | 7269.2 | mg |
| Glucose Ph. Eur. | 5532.5 | mg |
| Maracuja flavoring | 96.9 | mg |
| Water (Wfi) | 50503.7 | mg |

### EXAMPLE 3

### Lactic Acid Formulation Having a Concentration of 0.5 mg/ml.

For the preparation of the drink solution, indibulin is dissolved in lactic acid 90% Ph. Eur. and subsequently diluted with a solution of glucose and maracuja flavoring in water.

60 ml of an aqueous drink solution of indibulin having a concentration of 1 mg/ml are obtained. The excipients glucose and maracuja flavoring are the taste enhancers which facilitate oral administration.

Composition of the Solution:

| | | |
|---|---|---|
| Indibulin | 60.0 | mg |
| Lactic acid 90% | 14538.4 | mg |
| Glucose Ph. Eur. | 11065.0 | mg |
| Maracuja flavoring | 193.8 | mg |
| Water (Wfi) | 101007.4 | mg |

### EXAMPLE 4

### Bioavailability of the Indibulin Lactic Acid Formulation

In comparative kinetic investigations on cynomolgus monkeys, it was possible to show that the bioavailability of indibulin in the lactic acid formulation according to Example 1 in a dosage of 10 mg/kg orally administered (“p.o.”) with an area under the plasma concentration time curve (“AUC”) 0-24 h of 1364 (ng h/ml) is markedly better than a suspension of micronized indibulin (10 mg/kg p.o.) in 0.5% Tylose with an AUC 0-24 h of 250 (ng h/ml).

In addition, formulations of indibulin in capsules (50 mg, 100 mg) in comparison with the lactic acid formulation according to Ex. 1 were investigated on the cynomolgus monkey after oral administration and the plasma levels obtained were evaluated. The results are summarized in Table 1.

TABLE 1

| | Mean ± SD | | | | | |
|---|---|---|---|---|---|---|
| Admin. | | Animal | AUC_{0-24*} | AUC_{0-24, norm*} | AUC_{0-36*} | AUC_{0-36, norm*} |
| route | Treatment | group | [ng · h/ml] | [ng · h/ml] | [ng · h/ml] | [ng · h/ml] |
| po | Capsule 0109-019/01 (50 mg) | 1a | 524 ± 628 | 429 ± 473 | 561 ± 695 | 455 ± 510 |
| | Capsule 0110-022/01 (50 mg) | 1b | 76.6 ± 114 | 82.1 ± 139 | 103 ± 113 | 109 ± 137 |
| | Capsule 0112-001/01 (100 mg)** | 1a | 1425 ± 943 | 622 ± 537 | 1788 ± 1027 | 782 ± 634 |
| | Capsule 0112-001/01 (100 mg)** + | 1b | 756 ± 149 | 445 ± 108 | 988 ± 219 | 580 ± 142 |
| | 60 ml tylose | | | | | |
| | po solution (10 mg/kg) in 10% | 1a | 1886 ± 1085 | 1886 ± 1085 | 2863 ± 1810 | 2863 ± 1810 |
| | lactic acid | | | | | |
| iv | IV solution (0.2 mg/kg)* in | 1b | 299 ± 85.4* | 14949 ± 4270* | — | — |
| | sol/prop | | | | | |

*Plasma samples from intravenously administered animals were only withdrawn until 4 hours and, thus, only AUC₀₋₄ could be calculated
**Four animals

### EXAMPLE 5

The bioavailability of different formulations (suspension in solutol-propanediol and as an aqueous lactic acid solution) was tested on the cynomolgus monkey. The test formulations were administered orally and intravenously as a single dose. The plasma levels were determined and the AUC₀₋₂₄ calculated and based on a 10 mg/kg dose (=AUC_{0-24,norm}).

The study results show that indibulin in a 1 mg/ml lactic acid formulation shows a markedly higher bioavailability than in a self-emulsifying system of solutol-propanediol.

The absolute bioavailability based on an i.v. injection is about three times higher at 37% for the lactic acid drink solution (1 mg/ml) than for the solutol-propanediol system (13.2%).

Plasma AUC and absolute bioavailability of indibulin as a drink solution in 10% lactic acid solution of solutol: propanediol (“sol/prop”)(3:1; parts by weight) after sd (“single dose”) administration to the cynomolgus monkey are reported in Table 2 below.

TABLE 2

| | | | | [ng · h/ml] | | |
|---|---|---|---|---|---|---|
| Dose[mg/kg] | Route | Solvent | AUC | Mean ± SD | N² | F* [%] |
| 10.0 | po | Sol/prop (3:1) | 0-36, norm | 1969 ± 1040 | 6 | 13.2 |
| 0.2 | iv | Sol/prop (3:1) | 0-36, norm | 10167 ± 3207 | 6 | — |
| 10.0 | po | 10% lactic acid | 0-36, norm | 2863 ± 1810 | 6 | 19.2 |
| 0.2 | iv¹ | Sol/prop (3:1) | 0-4, norm | 14949 ± 4270 | 6 | 100 |
| 5.0 | po | 10% lactic acid | 0-42, norm | 5545 ± 3648 | 6 | 37.0 |
| | | (1 mg/ml) | | | | |
| 7.5 | po | 10% lactic acid | 0-42, norm | 2471 ± 1742 | 5 | 16.5 |
| | | (1.5 mg/ml)** | | | | |
| 10.0 | po | 10% lactic acid | 0-42, norm | 2363 ± 1318 | 6 | 15.8 |
| | | (2 mg/ml) | | | | |

¹Plasma samples from iv administered animals were only withdrawn until 4 hours, thus, only AUC₀₋₄ could be calculated.
²N: Number of animals (male and female)
*F: relative bioavailability (based on 100% of 0.2 mg/kg iv doses)

### EXAMPLE 6

Since the lactic acid drink solution has to be prepared immediately before administration, it is necessary to make available a preparation set. In the simplest case, this consists of 3 bottles containing the individual components in each case. The active compound is in this case filled into the largest bottle (mixing bottle), which is closed with a screw cap. The lactic acid and the aqueous glucose solution can be filled into injection bottles; in this case the glucose solution should be sterile in order to guarantee sterility.

The drink solution is prepared by addition of the lactic acid 90% Ph. Eur. to the active compound in the mixing bottle, and the substance is dissolved by shaking. Subsequently, the aqueous glucose solution is added and a homogeneous, clear, yellow solution is obtained by shaking.

The example can be markedly simplified in its applicability by means of an appropriate container. In this context, the container should consist of 3 chambers, which by simple operation, e.g. rotation or pressing, makes possible combination of the components in the correct sequence.

### EXAMPLE 7

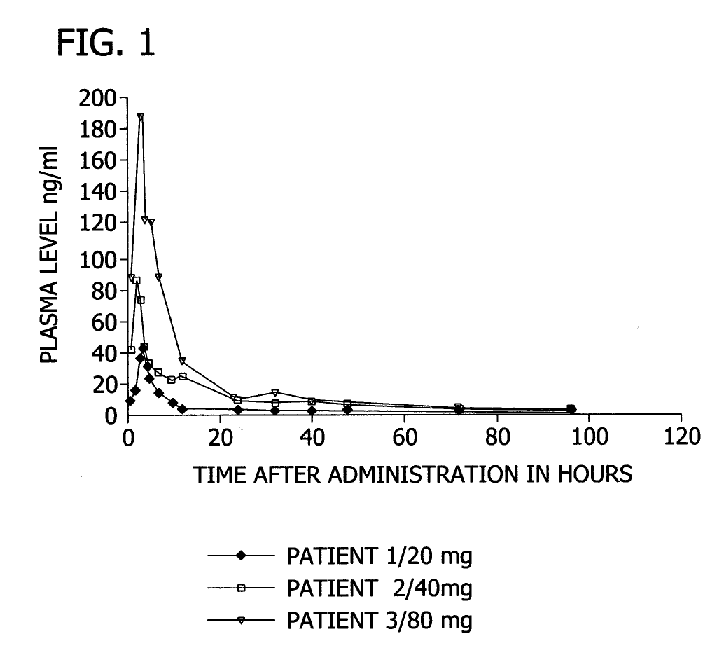

The formulation according to Example 1 was tested on humans in a phase 1 clinical study. In this study, doses of 20 mg, 40 mg and 80 mg were administered. The plasma level curves obtained are shown in FIG. 1.

What is claimed is:

1. A pharmaceutical presentation form comprising the poorly soluble active compound indibulin for oral administration, obtainable by dissolving indibulin in or with a highly concentrated organic acid which is physiologically tolerable per se in diluted form, and subsequent dilution by addition of water to a physiologically tolerable concentration of the organic acid, indibulin being present in the form of a supersaturated solution.

2. The pharmaceutical presentation form according to claim 1, wherein the organic acid is lactic acid.

3. The pharmaceutical presentation form according to claim 1, wherein the dissolving of the active compound takes place in an about 50% to about 90% by weight organic acid.

4. The pharmaceutical presentation form according to claim 1, wherein after diluting with water the concentration of the organic acid is less than about 20% by weight.

5. The pharmaceutical presentation form according to claim 4, wherein after diluting with water the concentration of the organic acid is in the range from about 5% to about 15% by weight.

6. The pharmaceutical presentation form according to claim 1, wherein after diluting with water the concentration of the active compound indibulin is in the range from about 0.2 mg/ml to about 2 mg/ml.

7. The pharmaceutical presentation form according to claim 6, wherein after diluting with water the concentration of the active compound indibulin is in the range from about 0.5 mg/ml to about 1.5 mg/ml.

8. The pharmaceutical presentation form according to claim 1, wherein optionally common excipients and additives are added.

9. The pharmaceutical presentation form according to claim 1, wherein an aqueous solution comprising taste ingredients is used for diluting.

10. the pharmaceutical presentation form according to claim 9, wherein the aqueous solution comprising taste ingredients is an aqueous solution of glucose and at least one flavoring.

11. The pharmaceutical presentation form according to claim 10, wherein the flavoring is maracuja flavoring.

12. The pharmaceutical presentation form according to claim 1, wherein the presentation form has a physical stability of about 2 hours.

13. The pharmaceutical presentation form according to claim 1, which is a drink solution.

14. A method for the preparation of a pharmaceutical presentation form comprising the poorly soluble active compound indibulin for oral administration, wherein the active compound indibulin is dissolved in a highly concentrated organic acid which is physiologically tolerable in diluted form, and subsequently the solution thus obtained is diluted by addition of water to a physiologically tolerable concentration of the organic acid, indibulin being present in the form of a supersaturated solution.

15. The method according to claim 14, wherein the organic acid is lactic acid.

16. The method according to claim 14, wherein the dissolving of the active compound is carried out in or with an about 50% to about 90% by weight organic acid.

17. The method according to claim 14, wherein after diluting with water the concentration of the organic acid is less than about 20% by weight.

18. The method according to claim 17, wherein after diluting with water the concentration of the organic acid is in the range from about 5% to about 15% by weight.

19. The method according to claim 14, wherein after diluting with water the concentration of the active compound indibulin is in the range from about 0.2 mg/ml to about 2 mg/ml.

20. The method according to claim 19, wherein after diluting with water the concentration of the active compound indibulin is in the range from about 0.5 mg/ml to about 1.5 mg/ml.

21. The method according to claim 14, wherein the pharmaceutical presentation form further comprises common excipients and additives.

22. The method according to claim 14, wherein an aqueous solution comprising taste ingredients is used for diluting.

23. The method according to claim 22, wherein the aqueous solution comprising taste ingredients is an aqueous solution of glucose and at least one flavoring.

24. The method according to claim 23, wherein the flavoring is a maracuja flavoring.

25. The method according to claim 14, wherein the presentation form thus prepared has a physical stability of about 2 hours.

26. The method according to claim 14, wherein the presentation form thus prepared is a drink solution.

27. A kit for the preparation of a pharmaceutical presentation form of claim 1, the kit comprising:
a specified amount of the active compound indibulin;
a specified amount of a highly concentrated organic acid; and
a specified amount of water.

28. The kit according to claim 27 further comprising taste ingredients.

29. The kit according to claim 27, wherein the organic acid is lactic acid.

30. The kit according to claim 27, wherein the highly concentrated acid has a concentration in the range from about 50% to about 90% by weight.

31. The kit according to claim 27, wherein the specified amount of water is calculated such that after diluting with water the concentration of the organic acid is less than about 20% by weight.

32. The kit according the claim 31, wherein the specified amount of water is calculated such that after diluting with water the concentration of the organic acid is in the range from about 5% to about 15% by weight.

33. The kit according to claim 27, wherein the specified amount of water is calculated such that after diluting with water the concentration of the active compound indibulin is in the range from about 0.2 mg/ml to about 2 mg/ml.

34. The kit according to claim 33, wherein the specified amount of water is calculated such that after diluting with water the concentration of the active compound indibulin is in the range from about 0.5 mg/ml to about 1.5 mg/ml.

35. The kit according to claim 27, wherein the water further comprises common excipients and additives.

36. The kit according to claim 35, wherein the water for diluting comprises one or more taste ingredients.

37. The kit according to claim 36, wherein the water for diluting comprises glucose and at least one flavoring.

38. The kit according to claim 37, wherein the water for diluting contains maracuja flavoring.

39. The kit according to claim 27, wherein the presentation form to be prepared thereby has a physical stability of about 2 hours.

40. The kit according to claim 27, wherein the presentation form to be prepared thereby is a drink solution.
